(19) United States
(12) Patent Application Publication
(10) Pub. No.: US 2003/0157581 A1
(43) Pub. Date: Aug. 21, 2003

(54) Use of an imaging photoelectric flat sensor for evaluating biochips and imaging method therefor

(76) Inventors: Hans-Horg Grill, Recklinghausen (DE); Norbert Leclerc, Freiburg (DE); Andreas Schutz, Oer-Erkenschwiek (DE); Lothar Prix, Reclinghausen (DE)

Correspondence Address: BIRCH STEWART KOLASCH & BIRCH, PO BOX 747, FALLS CHURCH, VA, 22040-0747, US

(21) Appl. No.: 10/333,774

(22) PCT Filed: Jul. 26, 2001
(86) PCT No.: PCT/EP01/08673

(30) Foreign Application Priority Data
Jul. 26, 2000 (DE) 100 36 457.8

(51) Int. Cl. C12Q 1/66

(52) U.S. Cl. 435/8

(57) ABSTRACT

The invention relates to the use of an image-generating photoelectric area sensor 14, for example a CCD sensor, for contact imaging the surface 11 of a biochip 13 by measuring a radiation emitted from the surface of said biochip, such as, for example, chemiluminescence, bioluminescence or fluorescence radiation, and to an image generation method therefor. For this purpose, the biochip 13 is arranged at a distance as short as possible from an area sensor 14. The area sensor 14 is then able, without insertion of an imaging optical system, to detect a spatially resolved two-dimensional image of the radiation emitted from the surface of the functionalized region 12.

The invention relates to the use of an image-generating photoelectric area sensor for evaluating biochips and to an image generation method therefor. The invention relates in particular to a method for the spatially resolved detection of electromagnetic radiation which is emitted by substances immobilized on a surface of a planar support, by means of an image-generating photoelectric area sensor.

The identification of particular genetic information is a fundamental objective in molecular biology, and a large number of very different methods have already been proposed in order to solve said objective. If an item of sought-after genetic information can be attributed to particular nucleic acid sequences (referred to as target sequences or targets hereinbelow), in many cases “oligonucleotide probes” are used whose nucleic acid sequences are complementary to the target sequences. Owing to their complementarity, said oligonucleotide probes and target sequences can hybridize in a specific manner so that it is possible to identify and analyze qualitatively and/or quantitatively the sought-after target sequences in a pool of extensive and complex genetic information.

Classical applications of this kind are Northern and Southern blots and also in-situ hybridization. For this purpose, the samples are usually prepared accordingly and investigated with the aid of defined oligonucleotide probes. In conventional applications of this kind, the oligonucleotide probes are usually labeled and can thus be detected, depending on the label chosen. This is necessary in order to be able to identify sample-bound probes, i.e. probes which have hybridized specifically to target sequences.

In order to label the probes, substances, i.e. markers, are used which can be identified with the aid of suitable detection methods. Common markers are in particular radioactive markers and also chemiluminescent or fluorescent markers.

Fluorescence and chemiluminescence methods, in particular, are highly regarded in chemical and biological analysis and diagnostics. These are very powerful detection methods which can be carried out without using radioactivity and, if necessary, without toxic substances. In comparison with radioisotopes, many of the markers used are virtually indefinitely stable when stored appropriately. There exist nowadays sensitive optical detection systems which make even detection of individual marker molecules possible. Moreover, there exists a large variety of very different fluorescent dyes so that it is possible to use fluorescent markers suitable for most wavelength ranges in the visible spectrum but also in the adjacent ultraviolet and infrared spectral regions. Accordingly, suitable chemiluminescence substrates are available for many enzymes, for example peroxidases, alkaline phosphatase, glucose oxidase and others. Powerful chemiluminescence substrates with a signal stability of more than one hour are commercially available.

In the above mentioned classical hybridization methods the number of different probes which can be used in connection with one and the same sample is limited. In order to be able to distinguish the probes, different, i.e. noninterfering labels and, consequently, also different detection systems are required. The expenditure connected therewith reaches, in multiple-parameter analyses at the latest, the limits of practicability.

In this respect, assay arrangements using immobilized oligonucleotide probes, i.e. probes attached to a solid support offer crucial advantages. In order to be able to detect in such systems the binding of sample and probe, the sample, not the probe, is labeled in these cases. In this connection, a solid support means a material having a rigid or semirigid surface. Possible examples of supports of this kind are particles, strands, in particular fiber bundles, spherical bodies such as spheres or “beads”, precipitation products, gels, sheets, tubes, containers, capillary tubes, disks, films or plates. However, the most common supports by now are planar, i.e. flat supports.

If a sample is to be investigated by means of a plurality of probes with different specificity, said probes are usually arranged on a shared support in such a way that each type of probe, i.e., for example, a particular oligonucleotide probe of a known sequence, is assigned to a particular field of a two-dimensional field pattern (generally referred to as “array”) on said support. Determining as to whether and/or, where appropriate, to what extent the labeled sample binds to a particular field, allows conclusions about the target sequence of said sample, which is complementary to the probe of said field, and possibly about the concentration thereof.

Since then, advances in miniaturization have made it possible to make the fields substantially smaller so that it is now possible to arrange a multiplicity of fields which are distinguishable in terms of method and measurement, i.e. also a multiplicity of distinguishable probes, on a single support. Although in the field of molecular biology glass supports are still the most common supports for these purposes, the planar supports are, following semiconductor technology, also referred to as “chips”, in particular as biochips, gene chips, etc. It is possible to bind the probes to the support with very high density and to arrange a plurality of probes of a single probe type in a miniaturized field. Currently, it is already possible to produce chips containing up to 40 000 different molecular probes per cm².

Especially the application of photolithographic manufacturing techniques from semiconductor technology has resulted in crucial advances in the production of such chips. The principle is based on a light-directed chemical solid-phase synthesis in which the fields are projected by photolithographic masks (cf., for example, Fodor et al., “Light-directed, spatially addressable parallel chemical synthesis”, Science, vol. 251, 767-773 (1991)). This method is particularly advantageous if the probes are to be synthesized from individual building blocks, for example nucleotides, in situ on the support. Thus it is possible to attach a particular building block specifically to the probes being synthesized of particular fields, while the probes of the remaining fields are left untouched. This is possible by using photolithographic masks which project light for the light-directed chemical synthesis only onto those fields to which the building block is to be attached. The incident light, for example, can cause light-sensitive protective groups to be cleaved off, thereby liberating a reactive group at exactly that site of the probes being synthesized to which the building block is to be attached. Since a building block attached last usually introduces a bound protective group and thereby protects again the probes extended by one building block, only a single building block is attached to an activated probe. For the same reason, the probes extended by one building block during a cycle as well as the probes not extended in said cycle are available as the initially protected entirety of all probes to a new specific activation by a suitable mask for the attachment of another building block in a new cycle. Methods of this kind are described in detail in international patent applications WO 90/15070, WO 91/07087, WO 92/10092, WO 92/10587, WO 92/10588 and in U.S. Pat. No. 5,143,854.

Other chips in turn have probes which are not synthesized in situ but are applied to the support in a prefabricated form. Corresponding arrays of biomolecules for analyzing polynucleotide sequences have already been described by E. Southern in international patent application WO 89/10977. Biomolecular arrays are suitable for a multiplicity of applications, starting from DNA sequencing via DNA finger printing to applications in medical diagnostics. By now, commercial biochips containing a multiplicity of different cDNAs for hybridization are already available. These cDNAs are, for example, nucleic acid sequences of approximately 200 to 600 base pairs (bp) in length, which are amplified by means of gene-specific primers, whose identity is checked by partial sequencing and which are then applied specifically to known locations, for example, on a nylon membrane.

Contacting the labeled samples with the planar chip can lead on individual fields to coupling, for example hybridization, with complementary probes. In those cases in which it is not expedient to label the samples, it is also possible to contact the chip with suitable labeled receptors which bind specifically to the samples, after the samples have bound to the probes. In both cases, fluorescent or chemiluminescent markers are immobilized on those field elements on which binding between probes and samples has taken place. In fluorescence-optical detection methods, the chip is then illuminated with light of a suitable wavelength so that the fluorescent dyes are excited and emit fluorescence radiation. In a method of detection by luminescence, no external excitation light is required. Rather, chemiluminescent or bioluminescent systems are used as markers for signal generation. The fluorescence or luminescence radiation generates a pattern of light and dark field elements on the planar support, which is recorded. Thus, information about the sample can be obtained by comparing the light/dark pattern with the known pattern of the biological probes attached to the support surface.

Advances in miniaturization have resulted in a large number of field elements on a single planar support, which, in commercial applications, must be measured very reliably in a short time. For the spatially resolved, fluorescence-optical detection of substances immobilized on a biochip, today mainly “scanners” are used which scan the surface of the chip using a focused laser beam and then detect the emitted fluorescence light. An appropriate fluorescence scanner is produced by Hewlett-Packard for evaluating Affymetrix biochips and is described in more detail in U.S. Pat. Nos. 5,837,475 and 5,945,679. Scanners in which a confocal excitation and detection system has been integrated into an epifluorescence microscope are also known. The systems used in scanners for detecting the emitted fluorescence light are usually “one-channel systems”, i.e., for example, individual photocells or secondary electron multipliers (photomultipliers).

However, two-dimensional detection systems such as, for example, CCD cameras, which can be used both for detecting fluorescence light and for detecting chemiluminescence light of a sample, are also known. Commercially available systems have either an optical imaging system which projects the biochip surface provided with chemiluminescent markers or fluorescent markers on a CCD sensor by using lens optics, or a combination of image intensifier and CCD camera.

Thus, DE 197 36 641 A1 describes an optical measuring system for biosensors, in which, for example, a CCD chip is used as detector. The object to be measured and the CCD chip are linked via optical equipment which may comprise fibers, lenses and mirrors.

U.S. Pat. No. 5,545,531 describes numerous detection systems for studying “biochip assays”, inter alia scanner systems and CCD systems with fast imaging optics. Also mentioned is the possibility of incorporating a CCD array into the waver of a biochip plate, without, however, disclosing to the skilled worker clear and comprehensible technical teaching on this matter.

U.S. Pat. No. 5,508,200 describes the evaluation of chemical assays by means of a video camera provided with imaging optics.

International patent application WO 97/35181 (PCT/US97/04377) describes an immunoassay system in which fluorescently labeled molecules of a biosensor are excited by evanescent light and the fluorescence radiation emitted by these molecules is registered by an array of photodetectors. In order to separate optically fluorescence from different regions of the biosensor, the fluorescence radiation is directed from the biosensor to the photodetectors assigned to the particular regions via “tunnels” with light-proof walls.

The known fluorescence or luminescence detection systems for biochips, however, have disadvantages. Thus, CCD cameras with lens optics are usually quite expensive, since either aspherical lenses which have been corrected in a complicated manner are used or, when using less complicated imaging optics, image distortion and vignetting needs to be corrected using complex image editing software. Moreover, in order to achieve sufficiently high sensitivity, usually cooled CCD sensors or “slow-scan, full-frame scientific chips” must be employed. Apart from high costs, the use of image intensifiers, too, is associated with further disadvantages. Thus, operation of an image intensifier requires a high-voltage connection on the imaging optics, leading to risks for the user when working with aqueous media such as, for example, buffers. If fiber optics are used, losses occur when coupling the fluorescence or luminescence light into or out of the fibers. Moreover, the resolution of fiber-optical imaging systems is limited. The imaging system of the above mentioned WO 97/35181, which consists of “light tunnels”, has the additional disadvantage that only beams of light which run essentially parallel to the tunnel axis can reach the detector. Moreover, the individual biosensor fields, the “light tunnels” and the detectors must be precisely aligned.

The present invention is therefore based on the technical problem of providing a simple and cost-effective image-generating system for spatially resolved detection of electromagnetic radiation, in particular luminescence and/or fluorescence radiation, which is emitted by substances immobilized on a planar surface of a support, in particular of a biochip. Said image-generating system should be simple to handle.

In order to solve this problem, the invention proposes using an image-generating photoelectric area sensor for contact imaging of a surface of a biochip. Surprisingly, it was found that it is possible to detect the fluorescence or luminescence radiation which is emitted by substances immobilized on an essentially planar surface of a biochip with spatial resolution and high sensitivity by arranging an image-generating photoelectric area sensor at a very short distance from the surface of the biochip, exciting the immobilized substances in order for them to emit electromagnetic radiation, preferably of light in the visible and/or infrared and/or ultraviolet spectral region, and detecting photoelectrically the emitted radiation without using an imaging optical system. In this connection, an “imaging optical system” means any equipment which radiates electromagnetic radiation which originates from a region of the surface of the biochip in an unambiguous manner to a particular region of the area sensor, i.e. in particular lens and mirror systems, gradient lens arrays, fiber bundles or light waveguide bundles, but also an arrangement of a plurality of “light tunnels” as described in WO 97/35181. Rather, in accordance with the present invention, a thin transparent plate (for example a glass plate) and/or a thin fluidic gap (for example a gap of air or liquid), at most, is provided between the surface of the biochip and the surface of the area sensor, because, surprisingly, it was found that, if the distance between biochip and area sensor is sufficiently short, a defined region of the biosensor is assigned to each photoelectric element of the area sensor, and the corresponding element need not be protected against scattered light from neighboring regions of the area sensor.

The term “excitation” here means not only excitation by irradiating with electromagnetic radiation, as is required, for example, for fluorescence detection. Rather, the term “excitation” is intended to comprise any influencing of the immobilized substances which is connected to the subsequent emission of light, in particular on the basis of chemiluminescence or bioluminescence. If “immobilized substances” are mentioned here, then this does not imply that the corresponding substances are completely immobile. Rather, this should express the fact that the mobility of the probes within an incubation and/or measurement interval, i.e. in the second or minute range, is so small that unambiguous spatial assignment of the substance to a field element of the biochip is still possible.

The present invention has numerous advantages:

The detection system of the invention is particularly cost-effective, since it does not need any lens optics, image intensifiers or fiber optics to project the biochip onto the area sensor. Conventional systems which employ optics must deal with high losses of signal due to said optics. Moreover, due to the smaller solid angle of the optical imaging systems, only a fraction of the starting signal reaches the detector over a relatively long distance. In the known detectors, these losses must be compensated for with expensive high-performance detectors or electronic image intensifiers. In contrast, it is possible in the present invention to receive emitted light from a large solid angle area, due to dispensing with any imaging optics and to the immediate spatial proximity of signal generation and detection. It is therefore possible to use simpler and cost-effective detectors. Moreover, the high signal yield makes possible very short measurement times; in some cases, a measurement time of less than 50 ms is sufficient for a complete biochip.

Compared to the prior art, the detection system is more compact, miniaturized to a high degree and simple to operate, since there is no need for focusing or adjusting. Dispensing with an optical imaging system makes it also impossible for image apparitions such as vignetting, distortion or change in dynamics to occur. Owing to its compactness and its miniaturization, the detection system of the invention can readily be integrated into automated analysis systems.

The operation is substantially easier and faster than that of an X-ray film, with similar sensitivities. In addition, directly digitized data are obtained which can be processed further.

Complicated alignment and adjustment of the biochip to the area sensor are not required, since the individual spot can also be found and identified after data recording, using software.

Preference is given to using a diode or transistor array, a CCD sensor (e.g. a video sensor, full frame sensor or line sensor, a slow-scan scientific CCD or else a line transfer model) or a TFA image sensor as area sensor. TFA means “Thin Film on ASIC (Application Specific Integrated Circuit)”. TFA image sensors consist, for example, of a thin layer of amorphous silicon on an ASIC sensor. In this connection, line arrays, too, are to be included in the term “area sensor”, since, for example, linear line arrays always cover a particular area of the biochip, due to their finite transverse dimensions.

Accordingly, the detection system consists, according to a preferred embodiment, of an image-generating area sensor and a biochip which is placed directly on the sensor area for measurement. A spacer which defines, for example, a reaction space which, in the case of a chemiluminescence or bioluminescence detection method, can be filled with luminescence system components, generally reactants involved in the luminescence reaction, for example chemiluminescence substrate, or, if the substrate is attached to the chip surface, with enzyme solution may be arranged between area sensor and biochip. Activation of the substrate leads to chemiluminescence or bioluminescence radiation to be emitted and to be detected photoelectrically and with spatial resolution by the area sensor.

Typically, area sensors containing more than 10 000 pixels are used. The sensor area is preferably at least as large becomes the biochip surface to be projected and is usually from 40 to 100 mm². Since all pixels of the area sensor are illuminated at the same time, rapid measurements are possible across a large area at the same time. The area sensor is preferably oriented essentially parallel to the surface of the biochip but may otherwise be arranged in the detection system largely randomly, for example horizontally, vertically or in an “upside down” orientation.

The direct contact or the very short distance between area sensor and biochip surface corresponds to a type of contact exposure as is known from photographic films or-plates, but without having to deal with the specific disadvantages thereof: thus, conventionally, each image requires a new photographic film or a new photographic plate which then has to be developed and fixed in a complicated manner. Before the images recorded with conventional photographic films or photographic plates can be processed or evaluated on a computer, they still need to be digitized after development. In contrast, the signals provided by the image-generating photoelectric area sensor can be digitized and processed on a computer during illumination. The signal integration time is variable and can be chosen depending on the type of image sensor used or on the strength of the chemiluminescence signal and may, where appropriate, even be determined finally only during the ongoing measurement. Moreover, the area sensor or the entire detection system into which it is integrated can be washed and dried after a measurement and then be used again.

If an image sensor, for example a video CCD sensor with low dynamic range, is used, the measurement range can be extended to at least 10 bit by automatically varying the exposure time, using suitable control software. Scientific CCDs are available even with a measurement range from 12 to 18 bit. When using “locally adaptive TFA sensors”, it is even possible to increase the dynamic bandwidth of 70 dB, known from conventional CMOS or CCD technologies, to a dynamic bandwidth of 150 dB or more by separating the pixel information into two separate signals.

The spatial resolving power which can be achieved when contact imaging the surface of a biochip, is determined firstly by the size of the pixels of the area sensor and secondly by the distance of the biochip from the area sensor. If a reaction space for carrying out chemiluminescence or bioluminescence reactions is provided for between area sensor and biochip, a spatial resolving power of 20 μm or better is to be achieved. In those case in which a direct contact of area sensor and biochip can be realized, the resolving power corresponds to the size of the pixels of the sensor itself.

Biochips which may be selected are all formats which have a planar surface or in which the active substance is not immobilized in depressions which are deeper than the desired spatial resolution. The biochips have substances immobilized on a planar support surface, it being possible for said immobilized substances to be biological probes attached to said surface and/or samples bound to said probes. In this connection, the probes, the samples, the probes and the samples or, where appropriate, other substrate molecules binding to said probes or said samples may be labeled.

The following substances may be used as support materials: glass (standard glass, Pyrex glass, quartz glass), plastics, preferably of high purity and low intrinsic fluorescence (such as polyolefins, e.g. PE (polyethylene), PP (polypropylene), polymethylpentene, polystyrene, PMMA (poly(methyl methacrylate)), polycarbonate, Teflon), metals (such as gold, chromium, copper, titanium, silicon), oxidic materials or coatings (ceramics, aluminum-doped zinc oxide (TCO), silica, aluminum oxide). The support materials may be designed as membranes (such as polysaccharides, polycarbonate, Nafion), three-dimensional structures (such as gels, e.g. polyacrylamide, agarose, ceramics) or else moldings from above materials, such as films and dipsticks. For better adhesion, reduction of unspecific binding or for covalent coupling of the probes, it may be necessary to apply an intermediate layer or to preactivate the surface, for example by silanes (alkylsilanes, epoxysilanes, aminosilanes, carboxysilanes), polymers (polysaccharides, polyethylene glycol, polystyrene, polyfluorinated hydrocarbons, polyolefins, polypeptides), alkylthiols, derivatized alkylthiols, lipids, lipid bilayers or Langmuir-Blodgett membranes.

The probes are applied to the surface by pipetting, dispensing, printing, stamping or in situ synthesis (such as, for example, photolithographic techniques). Preference is given to applying different probes to the surface in a two-dimensional pattern. It is then possible to assign an unambiguous position on the surface to each probe. The probes may be coupled covalently, via adsorption or via physical/chemical interactions of the probes with the surface. Any known techniques may be employed.

Probes mean structures which can interact specifically with one or more targets (samples). Thus, biochip probes normally serve to investigate biological targets, in particular nucleic acids, proteins, carbohydrates, lipids and metabolites. Preference is given to using the following probes: nucleic acids and oligonucleotides (single- and/or double-stranded DNA, RNA, PNA, LNA, either pure or else in combination), antibodies (human, animal, polyclonal, monoclonal, recombinant, antibody fragments, e.g. Fab, Fab′, F(ab)₂, synthetic), proteins (such as allergens, inhibitors, receptors), enzymes (such as peroxidases, alkaline phosphatases, glucose oxidase, nucleases), small molecules (haptens): pesticides, hormones, antibiotics, pharmaceuticals, dyes, synthetic receptors or receptor ligands. Particularly preferred probes are nucleic acids, in particular oligonucleotides.

The present invention also relates to a method for the spatially resolved detection of electromagnetic radiation, in particular of chemiluminescence, bioluminescence and fluorescence radiation, which is emitted by substances immobilized on a planar surface of a support, which method comprises arranging an image-generating photoelectric area sensor at a short distance from the surface of said support, exciting the immobilized substances in order for them to emit electromagnetic radiation and detecting photoelectrically the emitted radiation without using an imaging optical system.

Preferably, a chemiluminescence and bioluminescence radiation emitted by the immobilized substances is detected. In this case, there is no need for irradiating with excitation light so that the detection system of the invention can be realized particularly cost-effectively. The detection of luminescence radiation also has the advantage of said radiation originating directly from the surface of the planar support and of no interfering scattered radiation being emitted from the reaction space covering the support or from the support itself.

For chemiluminescence or bioluminescence measurements, the system may be designed in such a way that just binding of the sample to the probe leads to the emission of light. In these cases, the system components required for the luminescence reaction are provided by the formation of a probe/sample complex. It is also possible to add, only after probe and sample have bound, in a further step components still required, for example a suitable chemiluminescence substrate, which are converted by samples, which are now themselves bound to the fixed probes, to give light-emitting products. The chemiluminescence radiation is preferably generated by enzymic reactions on the surface of the planar support. For this purpose, either a chemiluminescence substrate or an enzyme complex is attached to the support and a solution of the enzyme or of a chemiluminescence substrate is added. Conversion of the substrate leads to the emission of light. In any case, the substances immobilized on the biochip which are to be detected are usually provided with a luminescence marker, either directly (use of enzymes, for example horseradish peroxidase (POD) or enzyme substrates (e.g. luminol)) or via a multi-step process (introduction of a primary label such as biotin or digoxigenin (DIG) and subsequent incubation with luminescent markers such as POD-labeled streptavidin or anti-DIG). The last step usually comprises the addition of enzyme substrate solution or, if substrate molecules such as luminol have been used as markers, of enzyme solution. The use of enzymes as markers has the advantage of the enzymic reaction achieving an enormous amplification of the signal. Any chemiluminescent or bioluminescent systems can be used as markers for signal generation for biochip evaluation, for example alkaline phosphatase with dioxetane (AMPPD) substrates or acridinium phosphate substrates; horseradish peroxidase with luminol substrates or acridinium ester substrates; microperoxidases or metal porphyrin systems with luminol; glucose oxidase, glucose-6-phosphate dehydrogenase; or else luciferin/luciferase systems.

According to another embodiment of the invention, a fluorescence radiation emitted by the immobilized substances is detected. Compared with the enzymic chemiluminescence systems, fluorescent dyes are advantageous in that it is possible to carry out the measurement directly after introducing the marker. In contrast, enzyme or protein markers (for example the frequently used biotin/streptavidin complex) require a further incubation step which comprises introducing the enzyme marker and adding the substrate solution. However, fluorescence measurements require irradiation with excitation light. Since the area sensor and the biochip surface are arranged at only a short distance from one another, preference is given to using a support into which excitation light can be coupled, for example, via the back facing away from the support. The excitation light coupled in is then guided in the support with total reflection, and the substances immobilized on the surface of the support are excited by evanescent light. Obviously, a support material with a fluorescence as low as possible should be considered here. Scattered light fractions in the signal detected by the area sensor can be suppressed by using sensors with fast response times. When exciting with short light pulses, it is then possible to distinguish the scattered light fraction from the time-delayed fluorescence signal of interest by temporal discrimination.

Finally, it is also possible, according to the invention, to immobilize the substances to be studied directly on the photoelectric area sensor. In this case, the area sensor simultaneously serves as support for said substances. The support here may be, for example, a thin quartz layer which is provided as a protective layer directly on the photoelectric cells of the area sensor. In addition, the surface of the area sensor may also be coated in a suitable manner (for example, a hydrophobic surface can be generated by means of silanization).

The area sensor can be integrated into a flow cell. Additional equipment for the addition of substrate, for washing and for drying may be provided.

The present invention may be utilized, for example, for evaluating noncompetitive or competitive assay methods. In noncompetitive assays, the sample to be analyzed binds to the probe which has been immobilized beforehand on the surface of the biochip. The sample may be provided with a chemiluminescence marker beforehand. It is also possible for the sample first to bind to the fixed probe and then to be labeled in a second step (e.g. in primer extension or rolling cycle PCR). In all of these cases, a measuring signal is obtained which increases with the amount of sample molecules bound. It is also possible for the interaction of the sample with the probes immobilized on the surface to change the activity of the chemiluminescence-catalyzing enzyme (reduction, amplification, e.g. enzyme inhibition assays) and for this change to be recorded as measuring signal. Examples of noncompetitive assay methods, which may be mentioned, are hybridization reactions of PCR products or of labeled DNA/RNA with oligonucleotides or cDNA immobilized on the surface, or sandwich immunoassays. In competitive assay methods, a labeled substance is added to the sample, whose properties of binding to the probe immobilized on the surface are similar to those of the sample itself. A reaction in which sample and marker compete for the limited number of binding sites on the surface takes place. A signal is obtained which decreases with the amount of sample molecules present. Examples of this are immunoassays (ELISA) or receptor assays.

The present invention is described in more detail below, with reference to exemplary embodiments depicted in the attached drawings.

In the drawings,

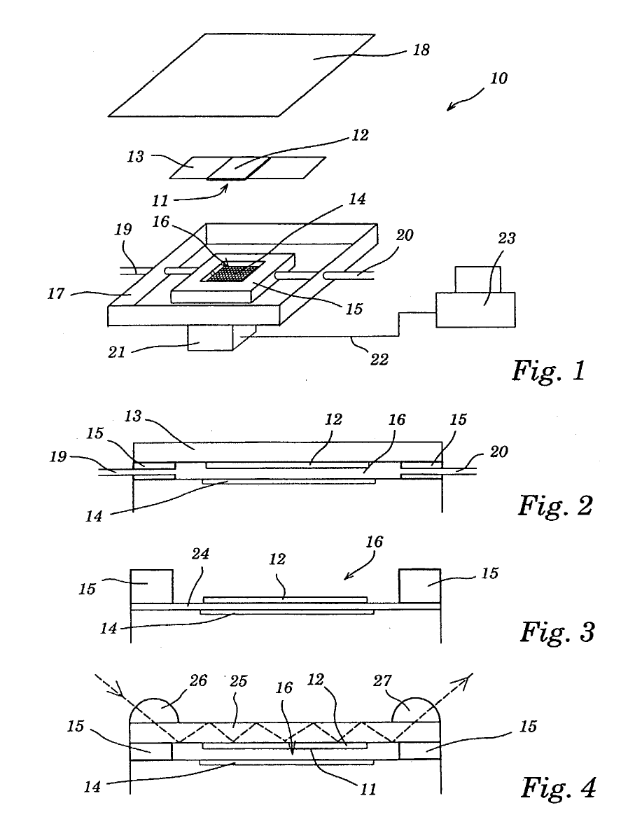

FIG. 1 shows a diagrammatic exploded illustration of an apparatus for contact imaging of the chemiluminescence radiation emitted by a biochip;

FIG. 2 shows a partial section of the arrangement of area sensor and biochip of the apparatus in FIG. 1;

FIG. 3: shows a partial section of an alternative arrangement of area sensor and biochip for detecting chemiluminescence radiation;

FIG. 4: shows an alternative arrangement of biochip and area sensor for detecting fluorescence radiation;

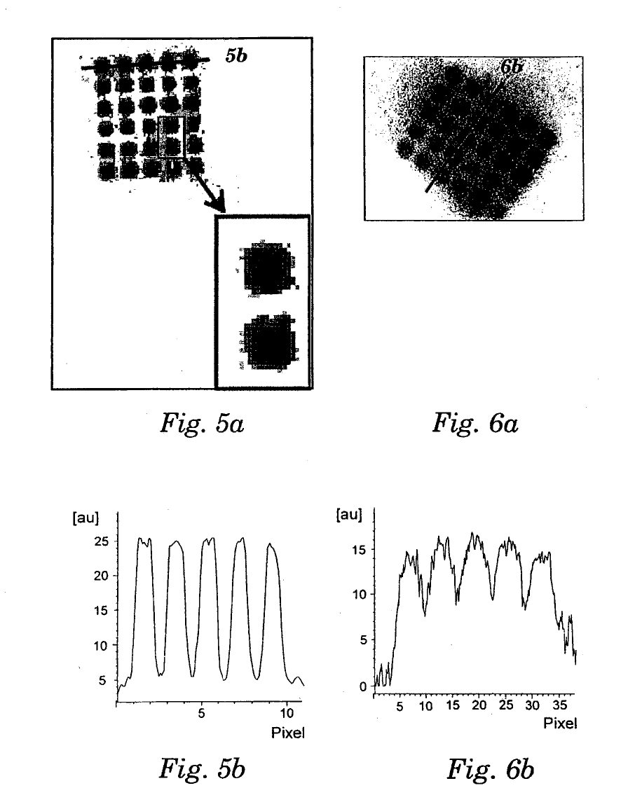

FIG. 5a: shows a CCD contact exposure image of the chemiluminescence radiation emitted by a biochip;

FIG. 5b: shows the intensity profile of the chemiluminescence signal along a line in FIG. 5a;

FIG. 6a shows an image of the biochip of FIG. 5a, obtained using X-ray film;

FIG. 6b shows the intensity profile along a line in FIG. 6a;

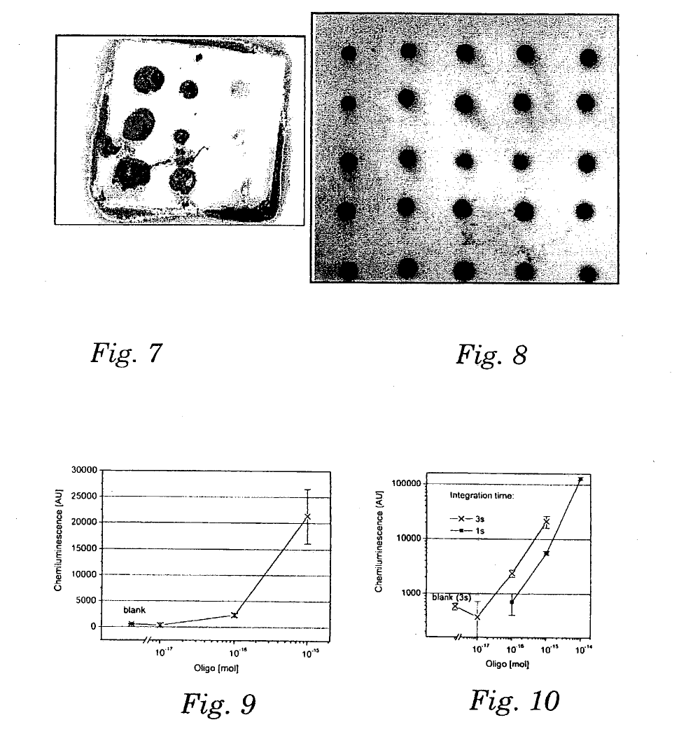

FIG. 7: shows a CCD contact exposure image of the chemiluminescence radiation emitted by a protein chip

FIG. 8: shows a CCD contact exposure image of the chemiluminescence radiation emitted by a DNA chip;

FIG. 9: shows a diagram representing the intensity of the chemiluminescence signal as a function of the immobilized oligonucleotide concentration;

FIG. 10: shows a diagram which indicates how to increase the range of measurement in the method of the invention by means of different exposure times;

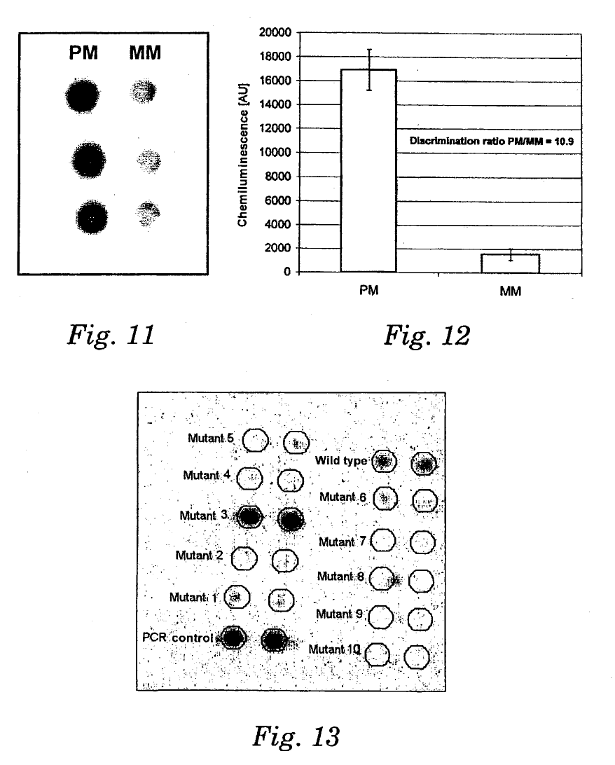

FIG. 11: shows a CCD contact exposure image of the chemiluminescence radiation emitted by another DNA chip;

FIG. 12: shows a diagram which illustrates a rate of discrimination determined from the image in FIG. 11;

FIG. 13: shows a CCD contact exposure image of a diagnostic biochip for determining mutations in oncogenes.

FIG. 1 depicts diagrammatically an exploded illustration of a detection system 10 for carrying out the method of the invention. In the example shown, the detection system 10 serves to measure chemiluminescence radiation with spatial resolution. Said chemiluminescence radiation is emitted by substances which are immobilized on the planar surface 11 of a functionalized region 12 of a biochip 13. For this purpose, the biochip 13 is arranged at a distance as short as possible from an area sensor 14, for example a CCD chip (cf. FIG. 2). The area sensor 14 is then able to detect, without insertion of an imaging optical system such as, for example, a lens or fiber optics, a spatially resolved, two-dimensional image of the chemiluminescence radiation emitted from the surface of the functionalized region 12. In the example depicted in FIG. 1, the biochip 13 rests on a washer 15 surrounding the area sensor of 14. The height of the washer 15 is chosen in such a way that, with the biochip in place, a gap left between the surface 11 of the functionalized region 12 and the area sensor 14 forms a reaction space 16 which can be filled, for example prior to placing the biochip 13, with a normally aqueous solution of a chemiluminescence substrate. The entire arrangement of biochip and area sensor is surrounded by a housing 17 which can be closed with a lid 18 in a light-tight manner. After placing the biochip 13, the chemiluminescence substrate is converted by enzymes immobilized in the functionalized region 12, resulting in the emission of light. The distance between the surface of the biochip 13 and the area sensor 14 is chosen so as for each pixel element of the sensor 14 to receive essentially only light from immediately opposite areas of the biochip. Therefore, the distance between area sensor and biochip should not substantially exceed the edge length of a pixel of the area sensor 14. Typically, said distance is thus in the range from 5-100 μm. In any case, the diameter of the individual field elements on the biochip itself must be regarded as the upper limit of said distance, since these field elements still need to be distinguished unambiguously from one another.

In a particularly simple embodiment of the apparatus for carrying out the method of the invention, the substrate solution can be introduced manually into the reaction space 16. In an automated arrangement, it is also possible to use, for example, pipetting robots for this purpose. However, it is also possible to fill or to flush the reaction space 16 with the aid of one or more lines 19, 20. In principle, the following automation steps can be carried out individually or in combination: placing or changing of the biochips 13, addition of substrate solution and, after measurement, washing and drying of the sensor 14. The area sensor may be integrated into a flow cell, in particular into an automated or manual flow injection system (FIA system) as part of a flow cell. The flow cell can be defined by area sensor and biochip by means of spacers.

The chemiluminescence light of the individual pixel elements which is detected by the area sensor 14 is digitized by means of an electronic control system 21 and transferred via a data line 22 to a computer 23 which also controls image recording, image processing and data storage. In a special embodiment, the computer is integrated directly into the system.

FIG. 2 depicts the arrangement of FIG. 1 in a partial section on a larger scale. The elements depicted are indicated by the same reference numbers as in FIG. 1.

FIG. 3 shows a variation of a measuring arrangement for detecting chemiluminescence radiation, in which the biochip consists of a thin film 24 which rests directly on the area sensor 14. The film 24 has, for example, a thickness of only 10 μm and is transparent for chemiluminescence light. This variant is advantageous in that the reaction spacer 16 can have any chosen depth, since it is located on that side of the film 24 which faces away from the sensor 14 and has therefore no influence on the resolving power of the detection system.

FIG. 4 depicts a measuring arrangement for detecting fluorescence light. The functionalized region 12 is located on a biochip 25 transparent for excitation light. Excitation light (indicated as a dashed line in FIG. 4) is coupled into the biochip 25, for example by means of two prisms 26, 27 glued onto opposite side edges of the support. The fluorescently labeled substances fixed on the surface 11 of the functionalized region 12 are excited by an evanescent portion of the excitation light and then emit fluorescence light which is subsequently recorded by the area sensor 14. If the excitation light and, consequently, also the emitted fluorescence light consist of short light pulses, a downstream electronic system can separate the signal recorded by the area sensor 14 into a possibly present scattered light portion and a slightly time-delayed emitted fluorescence portion actually of interest. For this reason, filtering equipment for removing the scattered light portion, arranged between biochip and area sensor, is not required.

### EXAMPLES

The following examples were carried out using a simple construction for manual operation, as is depicted diagrammatically in FIG. 1. An interline area sensor with video frequency is used (Sony, 768×576 pixel) CCD sensors of this kind are commercially available only in encapsulated form, i.e. the sensor is integrated in a housing made of ceramic and is closed at the top with a transparent glass cover. The area sensor was uncovered by removing the cover so that a short distance between sensor and biochip, which is required for a sharp image, can be realized. After uncovering the sensor, the electronic system was protected by sealing with a casting composition, in order to prevent short circuits when adding the aqueous substrate solution.

Addition of the substrate solution, placing of the biochip and, after measurement, washing with water and drying of the CCD sensor were carried out manually. The CCD sensor can be protected by applying to it a thin film (e.g. with a thickness of 3 μm) or a thin protective layer (e.g. coating layer). However, the thin SiO₂ layer usually present on a CCD chip is sufficient for protection against the aqueous substrate solution.

The read-out electronic system is housed in a camera module. The video signal is digitized by an 8 bit frame grabber in a computer. Controlling the on-chip integration achieves a considerable increase in sensitivity. Moreover, the dynamic range of the system can be expanded by means of different exposure times. The digitized image data can be stored directly in a common graphics format and are immediately available for further processing.

Controlling and image recording can be carried out directly in a memory chip of the detection system or externally via a PC or laptop.

### Example 1

### Optical Resolution

FIG. 5a depicts the image of a biochip as an example of the space-resolving power of the CCD sensor. The supports used were glass surfaces to which an array containing 5×6 field elements and made of a thin gold layer was applied very precisely. The surfaces were microstructured square gold surfaces with a side length of 100 μm and a center-to-center distance (grid) of 200 μm. The gold surfaces were biotinylated by treatment with an HPDP-biotin solution (Pierce). In order to saturate the entire surface with SH groups, the chips were treated in a second step with mercaptohexanol. It was now possible for streptavidin labeled with horseradish peroxidase (POD) (streptavidin-POD, Sigma, stock solution 1 mg/ml; dilution 1:10 000) to bind to the immobilized biotin. After a washing step and incubation with chemiluminescence substrate (SuperSignal Femto, ELISA, chemiluminescence substrate, Pierce), the chip was measured. For the image in FIG. 5a, the exposure time was set to 12 s. An inset in FIG. 5a depicts an enlarged illustration of two field elements so that even the individual pixels of the CCD sensor are visible. FIG. 5b depicts the profile of the chemiluminescence signal along the line “5b” in FIG. 5a. For comparative measurements, the biochip was measured by means of contact exposure using an X-ray film (Medical X-ray Film, Fuji RX, No. 036010). FIG. 6a depicts the result achieved using the X-ray film at an exposure time of likewise 12 s. FIG. 6b depicts the blackening curve of the film along the line “6b” in FIG. 6a. Comparison of FIGS. 5b and 6b, in particular, clearly indicates that it is possible to achieve good resolution of 100 μm structures using the measurement setup according to the invention and that the resolution obtained and the signal-to-noise ratio are better than when using the standard X-ray film. In this format, the sensitivity of the CCD chip corresponded to that of the X-ray film.

### Example 2

### Protein Chip

The surface of a glass slide was silanized with trimethylchlorosilane. An anti-peroxidase antibody (anti-peroxidase antibody, rabbit, Sigma) was immobilized by adsorption on this surface in individual spots and at different concentrations. After an incubation time of 3 h, the surface was blocked with a mixture of BSA and casein. The chips were then incubated with different concentrations of peroxidase (peroxidase from horseradish, grade I, Boehringer, stock solution 5 mg/ml) for 30-60 min, washed, admixed with chemiluminescence substrate and measured using the detection system (CCD chip, 3 μm film, 1.5 μl substrate solution, biochip placed). FIG. 7 depicts the result of the measurement. A protein chip with manually applied spots of 3 different anti-POD antibody concentrations (columns from left to right: dilution 1:100, 1:1 000, 1:10 000; POD dilution 1:10⁶; exposure time 35 s) is visible. In this assay format, the sensitivity was an order of magnitude higher than when using the X-ray film under identical conditions.

### Example 3

### DNA Chip

18-mer oligonucleotides (sequence: 5′ TATTCAGGCTGGGGGCTG-3′) were covalently immobilized on plastic supports. Hybridization was carried out using a complementary 18-mer probe which had been biotinylated at the 5′ end (5× SSP, 0.1% Tween, 1 h). A washing step was followed by incubation with streptavidin-POD (dilution 1:100; 5×SSP, 0.1% Tween 20; 30 min) and, after washing, by measurement using the setup already described in example 2. FIG. 8 depicts an overview of a biochip homogeneously spotted in an array of 5×5 field elements. It is also possible to add streptavidin-POD already to the hybridization solution. In this way, the number of incubation and washing steps is reduced and the assay is comparable to fluorescence systems with respect to performability and rapidity (apart from the addition of substrate).

The detection limit of detecting DNA was determined by hybridizing DNA chips at increasing concentrations to biotin probes, according to the above-described plan. FIG. 9 depicts the result. In this format (exposure time 1 s) the detection limit is at an absolute amount of DNA of below 10⁻¹⁶ mol and is limited by the background, i.e. the unspecific binding of streptavidin-POD to the immobilized oligonucleotides.

The dynamic range of the video-CCD chip used here of 8 bit can be compensated for by different exposure times. The diagram of FIG. 10 depicts the results of corresponding experimental studies. In this way it is possible to expand the measurement range to 10 bit and above.

As in other detection methods, the choice of stringent conditions (20 min of washing with 0.3×SSPE and 25% formamide after hybridization) makes it possible to discriminate well between perfect match (PM) samples and simple mismatch (MM) samples. For the above-mentioned immobilized 18-mer oligonucleotides, rates of discrimination of more than 10 are achieved when introducing a CC mismatch at position 7. FIG. 11 depicts the corresponding CCD contact exposure image of the resulting chemiluminescence signal of a DNA chip (spots in left-hand column: perfect match (PM); spots in right-hand column: mismatch (MM)). The diagram of FIG. 12 depicts the signal intensities. In the example depicted, the PM/MM intensity ratio is 10.9.

### Example 4

### Diagnostic Biochip—Determination of Mutations in Oncogenes

A DNA chip containing various 13-mer capture probes (immobilized probes) for 10 mutations of an oncogene was prepared. In addition, a probe for the wild type and a PCR control were integrated. DNA containing mutation 3 (see table below) was isolated from a cell line and amplified by means of mutation-enriching PCR (50 μl mixture containing approx. 10 ng DNA; 35 cycles; primer biotinylated at 5′ end; amplification length approx. 157 base pairs). The PCR product was adjusted to 6×SSPE using 20×SSPE and diluted 1:10 with 6×SSPE. Prior to hybridization, the mixture was admixed 1:1 with a 1:100 dilution of streptavidin-POD in 6×SSPE. The hybridization was followed by washing with 6×SSPE, addition of chemiluminescence substrate to the chip and measurement in the detector. The particular mutants (rates of discrimination of more than 20 compared with other mutants) can be unambiguously classified. FIG. 13 depicts the result of the corresponding chemiluminescence measurement (biochip after stringent hybridization (1 h, 37° C. 6×SSPE); 3 s exposure).

The following rates of discrimination were measured:

| Capture probe | Discrimination to mutant 3 |
|---|---|
| PCR control | 1.11 |
| Mutant 3 | 1.00 |
| Mutant 1 | 21.5 |
| Mutant 5 | 64.4 |
| Mutant 6 | 71.1 |
| Wild type | 10.5 |

For all other mutations, the discrimination is at >70.

1. The use of an image-generating photoelectric area sensor for contact imaging of the chemiluminescence radiation emitted by a surface of a biochip, the area sensor being integrated into a measuring cell which is designed as a flow cell.

2. The use as claimed in claim 1, characterized in that the area sensor is a diode array, a CCD sensor or a TFA image sensor.

3. A method for the spatially resolved detection of electromagnetic radiation which is emitted by substances immobilized on a surface of a support, which method comprises
arranging an image-generating photoelectric area sensor at a short distance from the surface of the support, so that a reaction space of a flow cell is defined between the area sensor and the support,
flushing the reaction space with a solution of enzyme or substrate in order to excite the immobilized substances so that they emit chemiluminescence or bioluminescence radiation, and
detecting photoelectrically the emitted radiation without using an imaging optical system.

4. The method as claimed in claim 3, characterized in that the sensor is washed and dried after the measurement.

5. The method as claimed in claim 4, characterized in that flushing, measuring, washing and drying are automated.

6. The method as claimed in either of claims 3 and 4, characterized in that chemiluminescence radiation emitted by the immobilized substances is detected.

7. The method as claimed in claim 6, characterized in that said chemiluminescence radiation is generated by enzymic reactions.

8. The method as claimed in either of claims 3 and 4, characterized in that bioluminescence radiation emitted by the immobilized substances is detected.

9. A method for the spatially resolved detection of electromagnetic radiation which is emitted by substances immobilized on a surface of a support, which method comprises
immobilizing said substances on a photoelectric area sensor used as support,
arranging said area sensor in a measuring cell designed as flow cell so that a reaction space is defined,
flushing the reaction space with a solution of enzyme or substrate in order to excite the immobilized substances so that they emit chemiluminescence or bioluminescence radiation, and
detecting photoelectrically the emitted radiation without using an imaging optical system.
